(19) United States
(12) Patent Application Publication
(10) Pub. No.: US 2004/0053251 A1
(43) Pub. Date: Mar. 18, 2004

(54) Methods of screening for alzheimer's disease

(76) Inventors: Margaret A. Pericak-Vance, Chapel Hill, NC (US); Jonathan L. Haines, Brentwood, TN (US)

Correspondence Address: MYERS BIGEL SIBLEY & SAJOVEC, PO BOX 37428, RALEIGH, NC, 27627, US

(21) Appl. No.: 10/311,668

(22) PCT Filed: Jun. 29, 2001
(86) PCT No.: PCT/US01/41224

(30) Foreign Application Priority Data
Jun. 30, 2000 (US) 60215151

(51) Int. Cl. C12Q 1/68

(52) U.S. Cl. 435/6

(57) ABSTRACT

Methods of screening a subject for Alzheimer's disease comprise detecting the presence or absence of a marker or functional polymorphism associated with a gene linked to Alzheimer's disease. The presence of such a functional polymorphism indicates that the subject is afflicted with or at risk of developing Alzheimer's disease.

### FIELD OF THE INVENTION

This invention concerns methods of screening for Alzheimer's disease, particularly late-onset Alzheimer's disease, by the screening of genetic risk factors.

### BACKGROUND OF THE INVENTION

Alzheimer's disease (AD) is a progressive degenerative disease of the central nervous system. It is characterized by progressive and increasing memory loss, followed by loss of control of limbs and bodily functions and eventual death. As the life expectancy in the United States and elsewhere has progressed, the number of individuals afflicted with Alzheimer's disease has grown accordingly. Currently, approximately 4 million Americans (one in five of those 75 to 84 years of age and nearly half of those 85 years old and older) are now afflicted. See Newsweek, pg 48 (Jan. 31, 2000).

U.S. Pat. No. 5,508,167 to Roses et al. describes the finding of a linkage of risk for Alzheimer's disease to the presence or absence of at least one Apolipoprotein E4 allele in an individual. Other techniques for screening for Alzheimer's disease are discribed in U.S. Pat. No. 5,297,562 to Potter and U.S. Pat. No. 5,972,638 to Tanzi et al. Nevertheless, the genetic basis for Alzheimer's disease is not well understood, and there is a continued need to develop new genetic linkages and markers and identify new functional polymorphisms that are associated with Alzheimer's disease.

### SUMMARY OF THE INVENTION

A method of screening a subject for Alzheimer's disease is described herein. The method comprises the steps of: detecting the presence or absence of a marker for Alzheimer's disease, or a functional polymorphism associated with a gene linked to Alzheimer's disease, with the presence of such a marker or functional polymorphism indicating that subject is afflicted with or at risk of developing Alzheimer's disease.

Of course, one, several, or all of the markers and/or functional polymorphisms associated with all of these genes may be screened in one individual, in one screening session or multiple screening sessions.

The detecting step may include detecting whether the subject is heterozygous or homozygous for the marker and/or functional polymorphism, with subjects who are at least heterozygous for the functional polymorphism being at increased risk for Alzheimer's disease.

The step of detecting the presence or absence of the marker or functional polymorphism may include the step of detecting the presence or absence of the marker or functional polymorphism in both chromosomes of the subject (i.e., detecting the presence or absence of one or two alleles containing the marker or functional polymorphism). Two copies of the marker or functional polymorphism (i.e., subjects homozygous for the functional polymorphism) may indicate greater risk of Alzheimer's disease as compared to heterozygous subjects.

A further aspect of the present invention is the use of a means of detecting a marker, functional polymorphism or mutation as described herein in screening a subject for Alzheimer's disease as described herein.

The foregoing and other objects and aspects of the present invention are explained in detail in the drawings herein and the specification set forth below.

### DETAILED DESCRIPTION OF THE PREFERRED EMBODIMENTS

As noted above, the present invention provides a method of screening (e.g., diagnosing or prognosing) for Alzheimer's disease in a subject. Subjects with which the present invention is concerned are primarily human subjects, including male and female subjects of any age or race.

The term “Alzheimer's disease” (AD) as used herein is intended to encompass all types of Alzheimer's disease, including sporadic and familial AD, as well as late onset and early onset AD.

The term “late-onset Alzheimer's disease” refers to Alzheimer's disease which has a time of onset after the subject reaches 40 years of age.

“Screening” as used herein refers to a procedure used to evaluate a subject for risk of idiopathic Alzheimer's disease. It is not required that the screening procedure be free of false positives or false negatives, as long as the screening procedure is useful and beneficial in determining which of those individuals within a group or population of individuals are at increased risk of idiopathic Alzheimer's disease. A screening procedure may be carried out for both prognostic and diagnostic purposes (i.e., prognostic methods and diagnostic methods).

“Prognostic method” refers to method used to help predict, at least in part, the course of a disease. For example, a screening procedure may be carried out on a subject that has not previously been diagnosed with Alzheimer's disease, or does not show substantial disease symptoms, when it is desired to obtain an indication of the future likelihood that the subject will be afflicted with Alzheimer's disease. In addition, a prognostic method may be carried out on a subject previously diagnosed with Alzheimer's disease when it is desired to gain greater insight into how the disease will progress for that particular subject (e.g., the likelihood that a particular patient will respond favorably to a particular drug treatment, or when it is desired to classify or separate Alzheimer's disease patients into distinct and different subpopulations for the purpose of conducting a clinical trial thereon). A prognostic method may also be used to determine whether a person will respond to a particular drug.

“Diagnostic method” as used herein refers to a screening procedure carried out on a subject that has previously been determined to at risk for a particular neurodegenerative disorder due to the presentation of symptoms or the results of another (typically different) screening test.

“Functional polymorphism” as used herein refers to a change in the base pair sequence of a gene that produces a qualitative or quantitative change in the activity of the protein encoded by that gene (e.g., a change in specificity of activity; a change in level of activity). The presence of a functional polymorphism indicates that the subject is at greater risk of developing a particular disease as compared to the general population. For example, the patient carrying the functional polymorphism may be particularly susceptible to chronic exposure to environmental toxins that contribute to Alzheimer's disease. The term “functional polymorphism” includes mutations.

A “present” functional polymorphism as used herein (e.g., one that is indicative of or a risk factor for Alzheimer's disease) refers to the nucleic acid sequence corresponding to the functional polymorphism that is found less frequently in the general population relative to Alzheimer's disease as compared to the alternate nucleic acid sequence or sequences found when such functional polymorphism is said to be “absent”.

“Mutation” as used herein sometimes refers to a functional polymorphism that occurs in less than one percent of the population, and is strongly correlated to the presence of a gene (i.e., the presence of such mutation indicating a high risk of the subject being afflicted with a disease). However, “mutation” is also used herein to refer to a specific site and type of functional polymorphism, without reference to the degree of risk that particular mutation poses to an individual for a particular disease.

“Linked” as used herein refers to a region of a chromosome that is shared more frequently in family members affected by a particular disease, than would be expected by chance, thereby indicating that the gene or genes within the linked chromosome region contain or are associated with a marker or functional polymorphism that is correlated to the presence of, or risk of, disease. Once linkage is established association studies (linkage disequilibrium) can be used to narrow the region of interest or to identify the risk conferring gene for Parkinsons' disease.

“Associated with” when used to refer to a marker or functional polymorphism and a particular gene means that the functional polymorphism is either within the indicated gene, or in a different physically adjacent gene on that chromosome. In general, such a physically adjacent gene is on the same chromosome and within 1 or 2 centimorgans of the named gene (i.e., within about 1 or 2 million base pairs of the named gene).

Markers (e.g., genetic markers such as restriction fragment length polymorphisms and simple sequence length polymorphisms) may be detected directly or indirectly. A marker may, for example, be detected indirectly by detecting or screening for another marker that is tightly linked (e.g., is located within 1 or 2 centimorgans) of that marker.

The presence of a marker or functional polymorphism associated with a gene linked to Alzheimer's disease indicates that the subject is afflicted with Alzheimer's disease or is at risk of developing Alzheimer's disease. A subject who is “at increased risk of developing Alzheimer's disease” is one who is predisposed to the disease, has genetic susceptibility for the disease or is more likely to develop the disease than subjects in which the detected functional polymorphism is absent. While the methods described herein may be employed to screen for any type of idiopathic Alzheimer's disease, a primary application is in screening for late-onset Alzheimer's disease.

The marker or functional polymorphism may also indicate “age of onset” of Alzheimer's disease, particularly subjects at risk for Alzheimer's disease, with the presence of the marker indicating an earlier age of onset for Alzheimer's disease.

Suitable subjects include those who have not previously been diagnosed as afflicted with Alzheimer's disease, those who have previously been determined to be at risk of developing Alzheimer's disease, and those who have been initially diagnosed as being afflicted with Alzheimer's disease where confirming information is desired. Thus it is contemplated that the methods described herein be used in conjunction with other clinical diagnostic information known or described in the art which are used in evaluation of subjects with Alzheimer's disease or suspected to be at risk for developing such disease.

The detecting step may be carried out in accordance with known techniques (see, e.g., U.S. Pat. Nos. 6,027,896 and 5,508,167 to Roses et al.), such as by collecting a biological sample containing DNA from the subject, and then determining the presence or absence of DNA encoding or indicative of the functional polymorphism in the biological sample (e.g., the Parkin gene exon 3 deletion mutation described herein). Any biological sample which contains the DNA of that subject may be employed, including tissue samples and blood samples, with blood cells being a particularly convenient source.

Determining the presence or absence of DNA encoding a particular functional polymorphism may be carried out with an oligonucleotide probe labelled with a suitable detectable group, and/or by means of an amplification reaction such as a polymerase chain reaction or ligase chain reaction (the product of which amplification reaction may then be detected with a labelled oligonucleotide probe or a number of other techniques). Further, the detecting step may include the step of detecting whether the subject is heterozygous or homozygous for the particular functional polymorphism. Numerous different oligonucleotide probe assay formats are known which may be employed to carry out the present invention. See, e.g., U.S. Pat. No. 4,302,204 to Wahl et al.; U.S. Pat. No. 4,358,535 to Falkow et al.; U.S. Pat. No. 4,563,419 to Ranki et al.; and U.S. Pat. No. 4,994,373 to Stavrianopoulos et al. (applicants specifically intend that the disclosures of all U.S. patent references cited herein be incorporated herein by reference).

Amplification of a selected, or target, nucleic acid sequence may be carried out by any suitable means. See generally D. Kwoh and T. Kwoh, Am. Biotechnol. Lab. 8, 14-25 (1990). Examples of suitable amplification techniques include, but are not limited to, polymerase chain reaction, ligase chain reaction, strand displacement amplification (see generally G. Walker et al., Proc. Natl. Acad. Sci. USA 89, 392-396 (1992); G. Walker et al., Nucleic Acids Res. 20, 1691-1696 (1992)), transcription-based amplification (see D. Kwoh et al., Proc. Natl. Acad Sci. USA 86, 1173-1177 (1989)), self-sustained sequence replication (or “3SR”) (see J. Guatelli et al., Proc. Natl. Acad Sci. USA 87, 1874-1878 (1990)), the Qβ replicase system (see P. Lizardi et al., BioTechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra). Polymerase chain reaction is currently preferred.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized which is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques, or by direct visualization on a gel. When PCR conditions allow for amplification of all allelic types, the types can be distinguished by hybridization with an allelic specific probe, by restriction endonuclease digestion, by electrophoresis on denaturing gradient gels, or other techniques.

DNA amplification techniques such as the foregoing can involve the use of a probe, a pair of probes, or two pairs of probes which specifically bind to DNA containing the functional polymorphism, but do not bind to DNA that does not contain the functional polymorphism. Alternatively, the probe or pair of probes could bind to DNA that both does and does not contain the functional polymorphism, but produce or amplify a product (e.g., an elongation product) in which a detectable difference may be ascertained (e.g., a shorter product, where the functional polymorphism is a deletion mutation). Such probes can be generated in accordance with standard techniques from the known sequences of DNA in or associated with a gene linked to Alzheimer's disease or from sequences which can be generated from such genes in accordance with standard techniques.

It will be appreciated that the detecting steps described herein may be carried out directly or indirectly. Other means of indirectly determining allelic type including measuring polymorphic markers that are linked to the particular functional polymorphism, as has been demonstrated for the VNTR (variable number tandem repeats) and the ApoB alleles (Decorter et al., DNA & Cell Biology 9(6), 461-69 (1990), and collecting and determining differences in the protein encoded by a gene containing a functional variant, as described for ApoE4 in U.S. Pat. No. 5,508,167 and 6,027,896 to Roses et al.

Kits for determining if a subject is or was (in the case of deceased subjects) afflicted with or is or was at increased risk of developing Alzheimer's disease will include at least one reagent specific for detecting for the presence or absence of at least one functional polymorphism as described herein and instructions for observing that the subject is or was afflicted with or is or was at increased risk of developing Alzheimer's disease if at least one of the functional polymorphisms is detected. The kit may optionally include one or more nucleic acid probes for the amplification and/or detection of the functional polymorphism by any of the techniques described above, with PCR being currently preferred.

While the present invention is described primarily in connection with the detection of Alzheimer's disease, it may be used to screen for other types of dementia as well.

Screening by Markers linked to Alzheimer's Disease. The present invention may be carried out by screening for markers within particular segments of DNA as described in (for example) U.S. Pat. No. 5,879,884 to Peroutka (the disclosure of which is incorporated by reference herein in its entirety. Examples of suitable markers, around which such segments may be identified, are given in Table 1-3 below.

In general, a method of screening for susceptibility to Alzheimer's Disease in a subject comprises determining the presence or absence of an allele of a polymorphic marker in the DNA of the patient, wherein (i) the allele is associated with the phenotype of Alzheimer's Disease, and wherein (ii) the polymorphic marker is set forth in Table 1-3 below, or a segment or region defined as being within 2, 5, 10, or 15 centiMorgans (cM) of the markers set forth in Table 1-3 below. The presence of the allele indicates the subject is at risk of developing Alzheimer's Disease.

To carry out the foregoing, nucleic acid samples can be collected from individuals of a family having multiple individuals afflicted with Alzheimer's Disease. Linkage within that family is then assessed within the regions set forth above in accordance with known techniques, such as have been employed previously in the diagnosis of disorders such as Huntington's disease, and as described in U.S. Pat. No. 5,879,884 to Peroutka. A disadvantage of such procedures is that the degree of confidence in the result may depend upon family size. Accordingly, another way to carry out the foregoing methods is to statistically associate alleles at a marker within the segments described above with Alzheimer's Disease, and use such alleles in genetic testing in accordance with known procedures.

Clinical trials and drug discovery. As noted above, the prognostic methods described herein may also be used to determine whether a person will respond to a particular drug. This is useful, among other things, for matching particular drug treatments to particular patient populations to thereby exclude patients for whom a particular drug treatment may be less efficacious.

Thus the present invention provides a computer assisted method of identifying a proposed treatment for Alzheimer's Disease (in a human subject). The method involves the steps of (a) storing a database of biological data for a plurality of patients, the biological data that is being stored including for each of said plurality of patients (i) a treatment type, (ii) at least one genetic marker associated with Alzheimer's Disease, and (iii) at least one disease progression measure for Alzheimer's Disease from which treatment efficacy may be determined; and then (b) querying the database to determine the dependence on said genetic marker of the effectiveness of a treatment type in treating Alzheimer's Disease, to thereby identify a proposed treatment as an effective treatment for a patient carrying a particular marker for Alzheimer's Disease.

In one embodiment, treatment information for a patient is entered into the database (through any suitable means such as a window or text interface), genetic marker information for that patient is entered into the database, and disease progression information is entered into the database. These steps are then repeated until the desired number of patients have been entered into the database. The database can then queried to determine whether a particular treatment is effective for patients carrying a particular marker, not effective for patients carrying a particular marker, etc. Such querying may be carried out prospectively or retrospectively on the database by any suitable means, but is generally done by statistical analysis in accordance with known techniques, as discussed further below.

Any suitable disease progression measure can be used, including but not limited to measures of motor function, measures of cognitive function, measures of dementia, etc., as well as combinations thereof. The measures are preferably scored in accordance with standard techniques for entry into the database. Measures are preferably taken at the initiation of the study, and then during the course of the study (that is, treatment of the group of patients with the experimental and control treatments), and the database preferably incorporates a plurality of these measures taken over time so that the presence, absence, or rate of disease progression in particular individuals or groups of individuals may be assessed.

An advantage of the present invention is the relatively large number of genetic markers for Alzheimer's Disease (as set forth herein) that may be utilized in the computer-based method. Markers as set forth in the prior art, including but not limited to those described in U.S. Pat. No. 5,508,167 to Roses et al., may also be used. Thus, for example, instead of entering a single marker into the database for each patient, two, three, five, seven or even ten or more markers (either including or excluding markers of the prior art, e.g., one, two, three, five, seven or even ten or more markers as set forth in Tables 1-3 herein, and those within 2, 5, 10 or 15 centimorgans thereof, and optionally including additional markers of the prior art such as ApoE), may be entered for each particular patient. Note that, for these purposes, entry of a marker includes entry of the absence of a particular marker for a particular patient. Thus the database can be queried for the effectiveness of a particular treatment in patients carrying any of a variety of markers, or combinations of markers, or who lack particular markers.

In general, the treatment type may be a control treatment or an experimental treatment, and the database preferably includes a plurality of patients having control treatments and a plurality of patients having experimental treatments. With respect to control treatments, the control treatment may be a placebo treatment or treatment with a known treatment for Alzheimer's Disease, and preferably the database includes both a plurality of patients having control treatment with a placebo and a plurality of patients having control treatments with a known treatment for Alzheimer's Disease

Experimental treatments are typically drug treatments, which are compounds or active agents that are parenterally administered to the patient (i.e., orally or by injection) in a suitable pharmaceutically acceptable carrier.

Control treatments include placebo treatments (for example, injection with physiological saline solution or administration of whatever carrier vehicle is used to administer the experimental treatment, but without the active agent), as well as treatments with known agents for the treatment of Alzheimer's Disease.

Administration of the treatments is preferably carried out in a manner so that the subject does not know whether that subject is receiving an experimental or control treatment. In addition, administration is preferably carried out in a manner so that the individual or people administering the treatment to the subject do not know whether that subject is receiving an experimental or control treatment.

Computer systems used to carry out the present invention may be implemented as hardware, software, or both hardware and software. Computer and hardware and software systems that may be used to implement the methods described herein are known and available to those skilled in the art. See, e.g., U.S. Pat. No. 6,108,635 to Herren et al. and the following references cited therein: Eas, M. A.: A program for the meta-analysis of clinical trials, Computer Methods and Programs in Biomedicine, vol 53, no. 3 (July 1997); D. Klinger and M. Jaffe, An Information Technology Architecture for Pharmaceutical Research and Development, 14^{th} Annual Symposium on Computer Applications in Medical Care, November 4-7, pp. 256-260 (Washington, D.C. 1990); M. Rosenberg, “ClinAccess: An integrated client/server approach to clinical data management and regulatory approval”, Proceedings of the 21^{st} annual SAS Users Group International Conference (Cary, N.C., Mar. 10-13, 1996). Querying of the database may be carried out in accordance with known techniques such as regression analysis or other types of comparisons such as with simple normal or t-tests, or with non-parametric techniques.

The present invention accordingly provides for a method of treating a subject for Alzheimer's Disease, particularly late-onset Alzheimer's Disease, which method comprises the steps of: determining the presence of a preselected marker for Alzheimer's Disease in said subject; and then administering to said subject a treatment effective for treating Alzheimer's Disease in a subject that carries said marker. The preselected marker is a marker such as described above, but to which a particular treatment has been matched. A treatment is preferably identified for that marker by the computer-assisted method described above. In one a particularly preferred embodiment, the method is utilized to identify patient populations, as delineated by preselected ones of markers such as described herein, for which a treatment is effective, but where that treatment is not effective or is less effective in the general population of Alzheimer's Disease patient (that is, patients carrying other markers, but not the preselected marker for which the particular treatment has been identified as effective).

The present invention is explained in greater detail in the following non-limiting Examples.

### EXAMPLE 1

### Identification of Genetic Risk Factors in Alzheimer's Disease

The purpose of the present study is to identify genetic risk factors in Alzheimer Disease (AD). Thus, we instituted a comprehensive genomic screen. We used a total of 466 families with late-onset (Table 1, family mean age of onset≧60 years) and over 400 microsatellite markers producing an approximate 7 cM grid. We designated as interesting any marker that resulted in a two-point lod score (MLS or parametric)≧1.00 (Table 1). Six regions, on chromosomes 4, 6q, 7, 9, 13, and 19 met this criterion. The results for D19S246 are detecting the effect of APOE, which is only 8 cM away. Linkage analysis with APOE itself generates lod scores approximately 3-fold stronger.

With a data set this large, it was possible for the first time to maintain power and still stratify the data into CONF (N=199) and UKN (N=267) families. These subsets have similar mean ages-at-onset (71.9 and 73.1 years, respectively, and 72.6 years for the combined (Comb) data set) and family size. As expected, however, the autopsy-confirmed group has a higher frequency of APOE-4 carriers among affecteds (77% vs 66%, P=0.02). Stratifying the data set produced an additional set of interesting results. The chromosome 4 and 13 results appear to come from both subsets, the chromosome 6q primarily from the unknown group, and the chromosome 7, 9, and 19 group primarily from the confirmed group. Several new regions come to light: chromosomes 5, 6, 10, and 18 in the confirmed group, and other regions on chromosomes 5 and 6 in the unknown group. The markers on chr 5 and 10 are in the same regions reported in Kehoe et al. (Kehoe, Hum. Mol. Genet. 8, 237-245 (1999)). Although there was some overlap between our two studies in the families analyzed from the NIMH data set, 51% of the families included in our screen are UNIQUE. There was no difference seen in the screen results with respect to the source of the study population (DUMC, IU, or NIMH).

Of particular importance is the result on chromosome 9. The peak MLS score of 2.97 in the combined data set increases to 4.31 in the stratified data set in the confirmed group. These results provide conclusive evidence for linkage to chromosome 9 in these data with the majority of the effect coming from the confirmed subset. (Lod≧3.00 is evidence of significant linkage.) It is also important to note that the positive results are spread across all three data sets (Duke, IU, and NIMH).

These data suggest that the CONF and UKN groups may well represent somewhat separate subgroups of dementia and that latter group may contain, in addition to classical AD (both APOE-4 related and unrelated), other clinical dementia subtypes. We are encouraged by these screening results since we are most likely searching for genes of more moderate effect that APOE. Our ability to look within these subsets at these regions will be extremely advantageous for fine mapping.

TABLE 1
Summary of Analyses for the Autopsy-Confirmed versus Unknown-Confirmed Families: Post NIMH Confirmation Update.

| | | | | | | 2 Point Affecteds Only Lod | | | 2 Point Affecteds Only Lod | | |
|---|---|---|---|---|---|---|---|---|---|---|---|
| | Marshfi | | ASPEX | | | Score-Dominant Model | | | Score-Recessive Model | | |
| Marker | eld cM | Cyto | Conf | Unknown | Combined | Conf | Unknown | Combined | Conf | Unknown | Combined |

| | | | | | | | | | | | |
|---|---|---|---|---|---|---|---|---|---|---|---|
| D4S1629 | 158 | 4q32.1 | 0.84 | 0.47 | 1.30 | 0.72 | 0.60 | 1.32 | 0.51 | 0.48 | 0.99 |
| D5S2849 | 8 | 5p15.3 | 0.00 | 0.95 | 0.42 | 0.06 | 0.59 | 0.47 | −0.07 | 1.01 | 0.49 |
| D5S1470 | 45 | 5p15.2 | 0.94 | 0.00 | 0.42 | 1.25 | 0.00 | 0.89 | 2.23 | −0.02 | 0.86 |
| D6S470 | 18 | 6p23-25 | 0.00 | 0.95 | 0.27 | −0.04 | 0.85 | 0.28 | −0.08 | 1.31 | 0.61 |
| D6S503 | 185 | 6q26 | 0.31 | 0.06 | 0.32 | 1.06 | 0.23 | 1.18 | 0.23 | 0.14 | 0.07 |
| D6S1027 | 187 | 6q26 | 1.03 | −0.01 | 0.56 | 0.90 | 0.01 | 0.60 | 1.20 | 0.00 | 0.52 |
| D7S2847 | 125 | 7q31.31 | 1.41 | 0.31 | 1.56 | 1.49 | 0.36 | 1.85 | 2.18 | 0.24 | 1.39 |
| D9S741 | 43 | 9p22.1 | 4.31 | 0.08 | 2.97 | 3.04 | 0.25 | 2.61 | 3.64 | 0.19 | 3.10 |
| D9S1818 | 151 | 9q34.2 | 1.96 | 0.00 | 0.19 | 2.05 | −0.17 | 0.44 | 2.04 | −0.36 | 0.38 |
| D10S1426 | 59 | 10p11.23 | 0.65 | 0.02 | 0.50 | 1.04 | −0.04 | 0.52 | 1.22 | −0.10 | 0.51 |
| D13S787 | 9 | 13q11- | 0.74 | 0.13 | 0.77 | 0.39 | 0.66 | 1.05 | 0.41 | 0.26 | 0.60 |
| 12.1 | | | | | | | | | | | |
| D18S878 | 99 | 18q22.1 | 0.32 | 0.00 | 0.00 | 0.65 | −0.30 | −0.07 | 1.02 | −0.55 | −0.01 |
| D18S1371 | 116 | 18q22.1 | 0.64 | 0.00 | 0.00 | 0.96 | −0.34 | 0.01 | 1.14 | −0.58 | 0.01 |
| D19S246 | 78 | 19q13.3 | 2.09 | 0.43 | 2.21 | 3.64 | 0.63 | 3.23 | 2.52 | 0.44 | 2.82 |
| APOE | 70 | 19q13.2- | 3.42 | 2.18 | 5.68 | 8.09 | 3.64 | 11.38 | 8.98 | 3.03 | 11.85 |
| | | 13.4 | | | | | | | | | |

Notes:
Conf n-199, Unknown n = 267, Duke n = 60, NIMH n = 286, IU n = 120

This new data set represents a significant opportunity to examine other traits associated with Alzheimer disease, such as age-at-onset (AAO). AAO was modeled as a quantitative trait and was analyzed using SIBPAL2 (SAGE) and Mapmaker Sibs (MS). As before, we examined the entire data set and the autopsy-confirmed and unknown subsets. Results were declared as interesting if P values <0.01 (SIBPAL) or lod score >1.0 (MS) were observed for any analysis (Table 2). Two particularly interesting results occur on chromosomes 11 and 21. The lower region on chr 11 maps near two excellent AD candidate loci, BACE and the amyloid precursor-like protein 2 (APLP2). The region on chromosome 21 maps near BACE2. In both cases, the results derive primarily from the UKN group. Multipoint SIBPAL analysis of these two regions provides even stronger evidence of linkage (Table 3). Again, the results derive almost exclusively from the autopsy-unknown group. These data further support the idea that this subset may be affected by a separate set of genes than the autopsy-confirmed subset.

Another interesting multipoint result is on chromosome 14 for marker D14S587 near the gene for Presenilin 1. MS multipoint analysis results in a lod score >1.00 in the average data set. There is a single convergence of chromosomal locations for both Alzheimer disease and Age-At-Onset. This occurs on chromosome 5 in the autopsy-unknown group near markers D5S2849 and D5S807.

TABLE 2
Interesting Results for Age-At-Onset from Genomic Screen II

| Marker | cM from Pter | Conf | Unk | Comb |
|---|---|---|---|---|
| D5S807 | 19 | 0.68 | 0.009 | 0.18 |
| D8S1136 | 82 | 0.008 | 0.82 | 0.11 |
| D11S1392 | 43 | 0.86 | 0.0003 | 0.10 |
| D21S1440 | 37 | 0.82 | 0.004 | 0.20 |
| D21S1446 | 58 | 0.63 | 0.0001 | 0.01 |

TABLE 3
Multipoint Analysis of Age-At-Onset (smallest P values or lod > 1.0)

| Marker | cM from Pter | Conf | Unk | Comb |
|---|---|---|---|---|
| D11S2371 | 76 | 0.63 | 0.003 | 0.08 |
| D11S4464 | 123 | 0.24 | 0.01 | 0.03 |
| D11S912 | 131 | 0.45 | 0.01 | 0.06 |
| D14S587 | 59 | — | — | 1.28 |
| D21S2052 | 25 | 0.04 | 0.22 | 0.03 |
| D211440 | 37 | 0.14 | 0.02 | 0.01 |
| GATA188F04 | 41 | 0.14 | 0.02 | 0.01 |
| D211411 | 52 | 0.44 | 0.0001 | 0.01 |
| D211446 | 58 | 0.53 | 0.0003 | 0.01 |

An overview of chr 21 and 11 show several likely AD candidate genes lying within or near peak regions of linkage, while chr 5 shows, to date, few obvious candidates. Two obvious candidates on chr 21 are the Amyloid Precursor Protein (APP) gene, which has already been shown to be the disease gene in a subset of early onset AD families and is known to be heavily involved in plaque formation and the pathology of AD, ETS2 also suggested as potentially involved in AD, and beta-site APP-cleaving enzyme 2 (BACE2) (Saunders et al., Science 246, 1255 (1999)). BACE2 was recently identified as a BACE homolog and localized to 21q22.3, lying near peak regions of linkage close to D21S1411. BACE2 possesses 52% identity to BACE at the amino acid level and 68% similarity. Like BACE (see below), BACE2 is hypothesized to be a beta-secretase. While its exact function is unknown, BACE2 lies in the obligatory Down Syndrome (DS) region on chr 21 and it has been suggested that it may be involved in the deposition and elevation of Aβ in DS patients. Regions of linkage on chr 11 are broader, but include such candidate genes as LRP5, the BCL2 antagonist of cell death (BAD), BDNF, Fadd, Apolipoproteins C3, A1, and A4, and intriguingly both BACE and Amyloid beta A4 precursor-like protein 2 (APLP2). BACE is the recently identified beta-secretase that, along with the γ-secretase, is involved in the proteolytic cleavage of APP that generates Aβ (Vassar et al., Science 286, 735-741 (1999)). APLP2 shares highly conserved homologies to APP at the amino acid level and is considered the nearest relative to APP. The region on chromosome 14 maps very near two excellent candidate genes: E SR2 (estrogen receptor 2) and PS1 (presenilin). PS1 is the gene involved in early onset AD and estrogen therapy has been indicated as playing in risk in females with AD.

The chromosome 5p region also contains interesting candidates including KIAA0300 and DAB2. Our most interesting region is on chromosome 9 with its MLS score >4.00. The chromosome 9 region contains two interesting candidates including phospholipase A2 activating protein (PLAP) and tyrosine kinase (TEK).

To identify AD causative genes, we have begun developing SNPs in candidate genes on chromosomes 5, 9, 11, and 21 for association and linkage studies. We have initially focused primarily on chromosome 9 together with 11 and 21 due to the quality of sequencing and genomic data and the large number of candidate genes available for study. We have identified a polymorphism in the 3′ untranslated region of PLAP in 2/18 control samples via SSCP. The C allele was present in approximately 11% of control individuals. OLA was used to type study samples. A second polymorphism was identified in 1/15 control individuals in the 5′ region of PLAP using HPLC. We are currently in the process of sequencing individuals to identify the base-pair change. We are also sequencing from pac DNA in order to identify intron/exon boundaries and develop primers for detecting additional SNPs. We are in the process of developing Oligonucleotide Ligation Assay (OLA) for two published SNPs (P. Chagnon et al., Alzheimer's Research 2, 237 (1996); E Cook et al., Molecular Psychiatry 2, 247-250 (1997)) in TEK, and endothelial tyrosine kinase gene. On chromosome 21, we are initially studying known polymorphisms and developing SNPs in the coding region of three candidate genes, APP, BACE2, and ETS2. These genes are either known to be directly involved in the etiology of Alzheimer's (APP), shown to interact directly or indirectly with such genes (ETS2), or are related to other genes of interest (BACE2). We are focusing on developing multiple SNPs that cover the full length of the gene. Substantial cDNA and genomic sequence is available for all three genes. We have currently designed, and are testing in 24 individuals (48 chromosomes), 12 sets of primers encompassing exonic sequence for ETS2 and 8 exonic primer sets for BACE2. Additional intronic primers are also being tested. Ampliners are being examined for polymorphisms using Transgenomic Wave DHPLC technology. For ETS2 we have detected a complex series of nucleotide changes in exon 8, which will be genotyped in our AD data set using the OLA. We are also examining the chromosome 21 TCF8 gene, but have been slowed by close homology between AREB6 and TCF8 ambiguities in the localization of these genes and sequences. We are pursuing a similar strategy on chromosome 11 initially focusing on the BDNF, LRP5, BACE, and APLP2 genes.

In summary, identification of genetic risk factors in AD will have a major impact on prevention diagnosis and treatment. Using novel approaches never used before in genomic screen analysis in AD including stratification (confirmed versus unconfirmed) and age-of-onset in AD as the trait locus, we have identified several areas of interest in late onset AD. One of these regions on chromosome 9 is of particular interest because it provides unequivocal evidence (lod score >4.00) of linkage to this region. The strength of the lod score supports the hypothesis that our linkage is very close to the actual risk gene involved. This finding, which is more significant than our original linkage finding on chromosome 19 (Pericak-Vance et al 1991) resulting in APOE identification, also supports the hypothesis that there is another susceptibility gene involved in late onset AD equal to or greater than the effect of APOE.

The foregoing is illustrative of the present invention, and is not to be construed as limiting thereof. The invention is defined by the following claims, with equivalents of the claims to be included therein.

That which is claimed is:

1. A method of screening a subject for Alzheimer's disease, comprising the steps of:
detecting the presence or absence of a marker linked to Alzheimer's disease;
the presence of said marker indicating said subject is afflicted with or at risk of developing Alzheimer's disease;
said marker selected from the group consisting of:
(i) D4S1629, D5S2849, D5S1470, D6S470, D6S503, D6S1027, D7S2847, D9S741, D9S1818, D10S1426, D13S787, D18S878, D18S1371, D19S246,
(ii) D5S807, D8S1136, D11S1392, D21S1440, D21S1446,
(iii) D11S2371, D11S4464, D11S912, D14S587, D21S2052, D211440, GATA188404, D211441, D211446, and
(iv) markers within 2 centimorgans thereof.

2. A method according to claim 1, wherein said Alzheimer's disease is late-onset Alzheimer's disease.

3. A method according to claim 1, wherein said subject has previously been determined to be at risk for Alzheimer's disease.

4. A method according to claim 1, wherein said method is a prognostic method.

5. A method according to claim 1, wherein said method is a diagnostic mutation.

6. A method according to claim 1, wherein said detecting step is carried out by:
collecting a biological sample from said subject; and then
detecting the presence or absence of said mutation from said biological sample.

7. A method according to claim 1, wherein said marker is linked to risk of Alzheimer's disease.

8. A method according to claim 1, wherein said marker is linked to age of onset of Alzheimer's disease.

9. A method according to claim 1, wherein said marker is D9S741 or a marker within 2 centimorgans thereof.

10. method of screening for susceptibility to Alzheimer's Disease in a subject, the method comprising:
determining the presence or absence of an allele of a polymorphic marker in the DNA of the patient, wherein (a) the allele is associated with the phenotype of Alzheimer's Disease, and wherein (b) the polymorphic marker is selected from the group consisting of:
(i) D4S1629, D5S2849, D5S1470, D6S470, D6S503, D6S1027, D7S2847, D9S741, D9S1818, D10S1426, D13S787, D18S878, D18S1371, D19S246,
(ii) D5S807, D8S1136, D11S1392, D21S1440, D21S1446,
(iii) D11S2371, D11S4464, D11S912, D14S587, D21S2052, D211440, GATA188404, D211441, D211446, and
(iv) markers within 2 centimorgans thereof,
the presence of said allele indicating said subject is at risk of developing Alzheimer's Disease.

11. The method according to claim 10, wherein said Alzheimer's Disease is late-onset Alzheimer's Disease.

12. The method according to claim 10, wherein said subject has previously been determined to be at risk for Alzheimer's Disease.

13. The method according to claim 10, wherein said method is a prognostic method.

14. The method according to claim 10, wherein said method is a diagnostic method.

15. A method according to claim 1, wherein said marker is D9S741 or a marker within 2 centimorgans thereof.

16. A computer assisted method of identifying a proposed treatment for Alzheimer's Disease, comprising the computer assisted steps of:
(a) storing a database of biological data for a plurality of patients, the biological data including for each of said plurality of patients (i) a treatment type, (ii) at least one genetic marker associated with Alzheimer's Disease, and (iii) at least one disease progression measure for Alzheimer's Disease from which treatment efficacy may be determined; and then
(b) querying said database to determine the dependence on said genetic marker of the effectiveness of a treatment type in treating Alzheimer's Disease, to thereby identify a proposed treatment as an effective treatment for a patient carrying a particular marker for Alzheimer's Disease.

17. The method according to claim 16, wherein said marker is the presence or absence of an allele of a polymorphic marker in the DNA of the patient, wherein (a) the allele is associated with the phenotype of Alzheimer's Disease, and wherein (b) the polymorphic marker is selected from the group consisting of:
(i) D4S1629, D5S2849, D5S1470, D6S470, D6S503, D6S1027, D7S2847, D9S741, D9S1818, D10S1426, D13S787, D18S878, D18S1371, D19S246,
(ii) D5S807, D8S1136, D11S1392, D21S1440, D21S1446,
(iii) D11S2371, D11S4464, D11S912, D14S587, D21S2052, D211440, GATA188404, D211441, D211446, and
(iv) markers within 2 centimorgans thereof;
the presence of said allele indicating said subject is at risk of developing Alzheimer's Disease.

18. A method according to claim 17, wherein said marker is D9S741 or a marker within 2 centimorgans thereof.

19. The method according to claim 16, wherein treatment type is selected from the group consisting of control treatments and experimental treatments.

20. The method according to claim 16, wherein said database includes a plurality of patients having control treatments and a plurality of patients having experimental treatments.

21. The method according to claim 16, wherein said control treatment is selected from the group consisting of placebo treatments and treatments with a known treatment for Alzheimer's Disease.

22. The method according to claim 16, wherein said database includes a plurality of patients having control treatment with a placebo, a plurality of patients having control treatments with a known treatment for Alzheimer's Disease, and a plurality of patients having experimental treatments.

23. The method according to claim 16, wherein said at least one disease progression measure is selected from the group consisting of tremor measures, rigidity measures, and akinesia measures.

24. The method according to claim 16, wherein said biological data for each of said plurality of patients includes at least three distinct genetic markers associated with Alzheimer's Disease.

25. The method according to claim 16, wherein said biological data for each of said plurality of patients includes at least five distinct genetic markers associated with Alzheimer's Disease.

26. The method according to claim 16, wherein said biological data for each of said plurality of patients includes at least ten distinct genetic markers associated with Alzheimer's Disease.

27. A method of treating a subject for Alzheimer's Disease, comprising the steps of:
determining the presence of a preselected marker for Alzheimer's Disease in said subject; and then
administering to said subject a treatment effective for treating Alzheimer's Disease in a subject that carries said marker,
and wherein said treatment is identified by the method of claim 16.

28. The method according to claim 27, wherein said Alzheimer's Disease is late-onset Alzheimer's Disease.
